(19) United States
(12) Patent Application Publication
(10) Pub. No.: US 2008/0031816 A1
(43) Pub. Date: Feb. 7, 2008

(54) Methods and compositions for identifying biomarkers

(76) Inventor: Charles Keller, San Antonio, TX (US)

Correspondence Address: MORGAN, LEWIS & BOCKIUS LLP (SF), 2 PALO ALTO SQUARE, 3000 El Camino Real, Suite 700, PALO ALTO, CA, 94306, US

(21) Appl. No.: 11/888,995

(22) Filed: Aug. 3, 2007

Related U.S. Application Data
(60) Provisional application No. 60/821,323, filed on Aug. 3, 2006.

(51) Int. Cl. A61K 49/00; A01K 67/00; A61P 43/00; C12Q 1/68; G01N 31/00; G01N 33/00; G01N 33/48; G01N 33/92

(52) U.S. Cl. 424/9.1; 435/6; 435/7.1; 436/104; 436/63; 436/71; 436/86; 800/3

(57) ABSTRACT

The present invention provides methods and compositions for the identification and validation of biomarkers. The methods and compositions of the invention include the use of exogenous molecules specifically designed and chosen to be associated with a particular disease or biological process. Biomarkers identified and used according to the invention can be indicative of a number of biological processes, including disease state, response to a therapeutic intervention (such as pharmacological treatment, radiation therapy, chemotherapy, combination therapies, and the like), and responses to physiological challenges (such as aging, environmental toxins, etc.). Biomarkers identified through the methods and compositions of the invention may also serve as targets of therapeutic interventions.

### CROSS-REFERENCE TO RELATED APPLICATIONS

The present application claims the benefit of the filing date of U.S. Provisional Patent Application No. 60/821,323, filed Aug. 3, 2006, which is incorporated by reference in its entirety for all purposes.

### FIELD OF THE INVENTION

The present invention relates generally to the identification and validation of diagnostic biomarkers.

### BACKGROUND OF THE INVENTION

Biomarkers (also called “biological markers”) are biological characteristics (e.g. enzyme concentration, hormone concentration, gene phenotype distribution in a population, presence of biological substances) that can be objectively measured and evaluated as an indicator of normal biological processes, of pathogenic processes, and of responses to a therapeutic intervention. Biological markers can reflect a variety of disease characteristics, including the level of exposure to an environmental or genetic trigger, an element of the disease process itself, an intermediate stage between exposure and disease onset, or an independent factor associated with the disease state but not causative of pathogenesis.

Biomarkers are powerful tools in medical research and drug discovery, as they often serve as signposts for diseases whose causative factors are not yet fully elucidated. In evaluating potential drug therapies, biomarkers can also be used as “surrogate endpoints” which are outcome measures that are not of direct practical importance but are believed to reflect clinically significant outcomes. For example, blood pressure is not directly important to patients but it is often used as an outcome in clinical trials because it is a risk factor for stroke and heart attacks. Surrogate endpoints are often physiological or biochemical characteristics that can be relatively quickly and easily measured and that are taken as being predictive of important clinical outcomes. They are often used when observation of clinical outcomes requires long follow-up. Biomarkers are of particular use as such surrogate endpoints.

Biomarkers can be naturally occurring or can be introduced into an organism to analyze and monitor a particular biological function. One example of naturally occurring biomarkers are single nucleotide polymorphisms (SNPs), which can be located near or within genes which are causative factors of disease. An example of biomarkers introduced into an organism is rubidium chloride, which is a radioactive isotope often used to study perfusion of heart muscle.

Despite their extensive utility, identifying useful biomarkers can be difficult. Genomics and proteomics applications, such as high throughput screening of nucleic acids, proteins, and/or clinical observations, will invariably result in a hundreds to thousands of potential biomarkers. However, most of these potential biomarkers will be false positives, meaning that they will not accurately or consistently be associated with the particular biological/disease state in which we are interested. An additional difficulty arises from the fact that some biomarkers are not present in large numbers within an organism, resulting in a low signal-to-noise ratio that can further complicate the identification process.

Any potential biomarker must be validated to determine whether it is a truly diagnostic biomarker, that is, whether it is consistently and detectably associated with a particular biological state or physiological response to stimulus. Traditional validation techniques generally require extensive time and resources, because they require an analytical test system with well established performance characteristics and for which there is widespread agreement in the medical or scientific community about the physiologic, toxicologic, pharmacologic, or clinical significance of the results.

Drug discovery and biomedical research applications require an efficient method for both identifying and validating biomarkers which can be used to diagnose disease and monitor the effects of therapeutic interventions.

### SUMMARY OF THE INVENTION

Accordingly, the present invention provides methods, compositions and systems for identifying and validating diagnostic biomarkers for downstream uses such as diagnosing disease and monitoring the effects of pharmacological and other therapeutic interventions.

In one aspect, the invention provides a method of identifying a diagnostic biomarker in a subject expressing a BioReporter. In a preferred aspect, the method includes the following steps: (i) comparing a property of a candidate biomarker at a first time point with that same property of the candidate biomarker at a second time point, thereby determining a comparison value for that property of the candidate biomarker; (ii) comparing a property of a BioReporter at a first time point with that same property of the BioReporter at said second time point, thereby determining a comparison value for that first property of the BioReporter; (iii) comparing the comparison value for the candidate biomarker with the comparison value for said BioReporter to determine a correlation for the comparison value for the candidate biomarker and the comparison value for the BioReporter; and (iv) comparing the correlation with a reference correlation, thereby identifying the candidate biomarker as a diagnostic biomarker.

In another aspect, the method provides a method of diagnosing a disease in a patient by determining a diagnostic biomarker property and analyzing that diagnostic biomarker property to determine a diagnostic biomarker property value. That diagnostic biomarker property value is then compared to a reference diagnostic biomarker property value in order to diagnose a disease in the patient. In a preferred aspect, the diagnostic biomarker is identified by a method including the steps of: (i) comparing a property of a candidate biomarker at a first time point with that same property of the candidate biomarker at a second time point, thereby determining a comparison value for that property of the candidate biomarker; (ii) comparing a property of a BioReporter at a first time point with that same property of the BioReporter at said second time point, thereby determining a comparison value for that first property of the BioReporter; (iii) comparing the comparison value for the candidate biomarker with the comparison value for said BioReporter to determine a correlation for the comparison value for the candidate biomarker and the comparison value for the BioReporter; and (iv) comparing the correlation with a reference correlation, thereby identifying the candidate biomarker as a diagnostic biomarker.

In another aspect, the invention provides a method of determining effectiveness of a treatment for a disease in a patient. In this aspect, the method includes the steps of determining a diagnostic biomarker property and analyzing that diagnostic biomarker property to determine a diagnostic biomarker property value. That diagnostic biomarker property value is then compared to a reference diagnostic biomarker property value in order to determine the effectiveness of the treatment. In a preferred aspect, the diagnostic biomarker is identified by a method including the steps of: (i) comparing a property of a candidate biomarker at a first time point with that same property of the candidate biomarker at a second time point, thereby determining a comparison value for that property of the candidate biomarker; (ii) comparing a property of a BioReporter at a first time point with that same property of the BioReporter at said second time point, thereby determining a comparison value for that first property of the BioReporter; (iii) comparing the comparison value for the candidate biomarker with the comparison value for said BioReporter to determine a correlation for the comparison value for the candidate biomarker and the comparison value for the BioReporter; and (iv) comparing the correlation with a reference correlation, thereby identifying the candidate biomarker as a diagnostic biomarker.

In another aspect, the invention provides a method of identifying a diagnostic biomarker in a subject. This aspect of the invention includes the step of inducing expression of a BioReporter in a subject. In a preferred aspect, this method further includes the steps of: (i) comparing a property of a candidate biomarker at a first time point with that same property of the candidate biomarker at a second time point, thereby determining a comparison value for that property of the candidate biomarker; (ii) comparing a property of a BioReporter at a first time point with that same property of the BioReporter at said second time point, thereby determining a comparison value for that first property of the BioReporter; (iii) comparing the comparison value for the candidate biomarker with the comparison value for said BioReporter to determine a correlation for the comparison value for the candidate biomarker and the comparison value for the BioReporter; and (iv) comparing the correlation with a reference correlation, thereby identifying the candidate biomarker as a diagnostic biomarker

In yet another aspect, the invention provides a kit. Kits of the invention can include: an isolated diagnostic biomarker, a container, and instruction for using the diagnostic biomarker. In a preferred aspect, the isolated diagnostic biomarker is prepared by a method which includes the steps of (i) identifying the diagnostic biomarker, and (ii) isolating the diagnostic biomarker. In a particularly preferred embodiment, the diagnostic biomarker is identified using a method which includes the steps of: i) comparing a property of a candidate biomarker at a first time point with that same property of the candidate biomarker at a second time point, thereby determining a comparison value for that property of the candidate biomarker; (ii) comparing a property of a BioReporter at a first time point with that same property of the BioReporter at said second time point, thereby determining a comparison value for that first property of the BioReporter; (iii) comparing the comparison value for the candidate biomarker with the comparison value for said BioReporter to determine a correlation for the comparison value for the candidate biomarker and the comparison value for the BioReporter; and (iv) comparing the correlation with a reference correlation, thereby identifying the candidate biomarker as a diagnostic biomarker.

In a preferred aspect, kits of the invention include a container, and the containers of the invention include the isolated diagnostic biomarker. In a further aspect, kits of the invention include instructions for using the diagnostic biomarker. Uses for the diagnostic biomarker include: diagnosing a disease, determining effectiveness of a treatment, and identifying causative factors of a disease.

In one aspect, the invention provides BioReporter systems. These BioReporter systems include organisms expressing one or more BioReporters. These BioReporter systems in a preferred embodiment include transgenic mice that have been genetically altered to express one or more BioReporters.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a graphical illustration of a squared errors assessment against data sets of a BioReporter and a candidate biomarker x.

FIG. 2 is a fold change color visualization assessment using a two-color system (FIG. 2A) and a four-color system (FIG. 2B).

FIG. 3 is an illustration of the conditional dual reporter allele (FIG. 3A) and the adeno-associated virus type 2 construct (AAVcre) (FIG. 3B).

FIG. 4 shows the time course of serial luminescence in Rosa26^{GoLUSAP/WT} HPRT^{Cre/WT} reporter mouse and wild type control following a single dose of Luciferin.

FIG. 5 shows the time course of focal luminescence in a Rosa26^{LUSAPm/WT} reporter mouse whose right vastus lateralis had been injected with an AAVcre three weeks prior to an intraperitoneal injection of luciferin. FIG. 5A shows the time course of the development of extraperitoneal luminescence following the luciferin injection. FIG. 5B shows the time course of luminescence measured by Total Flux. FIG. 5C shows the time course of luminescence measured by Average Radiance.

FIG. 6 shows the time course of dual marker signal intensity. Signal was compared between a wild type mouse, a mouse with Cre activation in the right thigh, a Rosa26^{LUSAPm/WT} Myf6^{ICNm/WT} mouse with Cre activation throughout all skeletal muscle, and a Rosa26^{GoLUSAP/WT} HPR^{Cre/WT} mouse with ubiquitous Cre activation. FIG. 6A shows a qualitative comparison of luminescence among the 4 mice. FIG. 6B shows the values total flux of luminescence, with a dynamic range of nearly 2.5 logs above background. Statistically significant differences are indicated with p-values. FIG. 6C shows values for serum alkaline phosphatase activity, with a dynamic range of more than 2 logs above background.

FIG. 7 shows a comparison of signal intensity of a monomeric red fluorescent protein reporter with a control mouse. FIGS. 7A and 7B show qualitative and quantitative fluorescence of a white-coated Z/RED-Tg^{GoZRED/WT} HPRT^{Cre/WT} monomeric red fluorescent protein reporter mouse with ubiquitous Cre activation. FIGS. 7C and 7D show the fluorescence from a shaved animal. FIGS. 7E and 7F show signal from the same animal shown in 7C and 7D with skeletal muscle Cre activation.

FIG. 8 illustrates a construct comprising a Pax7-Cre driver with spatial and temporal selectivity (FIG. 8A) and how tamoxifen induces recombination (FIG. 8B).

FIG. 9 shows Cre activity in the midface, midbrain, neural tub and somites of a mouse embryo before and after application of tamoxifen.

FIG. 10 illustrates a Lineage Tracing Experiment using a Cre Reporter Allele.

FIG. 11 shows activation of Pax3:Fkhr and LUSAP Dual Reporter by Pax7-CreER in a young adult mouse.

### DETAILED DESCRIPTION OF THE PREFERRED EMBODIMENTS

### Definitions

The singular forms “a,” “an,” and “the” include plural references, unless the context clearly dictates otherwise. Thus, for example, references to a composition including “a biomarker” encompass one, two or more biomarkers.

The term “biomarker” refers to any biological feature from an organism which is useful or potentially useful for measuring the initiation, progression, severity, pathology, aggressiveness, grade, activity, disability, mortality, morbidity, disease sub-classification or other underlying feature of one or more biological processes, pathogenic processes, diseases, or responses to therapeutic intervention. A biomarker is virtually any biological compound, such as a protein and a fragment thereof, a peptide, a polypeptide, a proteoglycan, a glycoprotein, a lipoprotein, a carbohydrate, a lipid, a nucleic acid, an organic on inorganic chemical, a natural polymer, and a small molecule, that is present in the biological sample and that may be isolated from, or measured in, the biosample. The concept of a biomarker also includes a physical measurement on the body, such as blood pressure, which is useful for measuring the initiation, progression, severity, pathology, aggressiveness, grade, activity, disability, mortality, morbidity, disease sub-classification or other underlying pathogenic or pathologic feature of one or more diseases. The concept of a biomarker also includes a pharmacological or physiological measurement which is used to predict a toxicity event in an animal or a human. A biomarker may also be the target for monitoring the outcome of a therapeutic intervention (e.g., the target of a drug agent).

A “diagnostic biomarker” as used herein is a biomarker which has been identified and validated as useful or potentially useful for use as a biomarker for a particular disease. This validation can be accomplished by a variety of techniques described herein, including correlation of a property, feature or characteristic of the biomarker with a property, feature, or characteristic of a BioReporter. A diagnostic biomarker can also be referred to as a “natural biomarker” or a “spontaneous biomarker”.

A “candidate biomarker” is a biomarker that has the potential to be a diagnostic biomarker but has not yet been validated by correlation to a BioReporter.

A “BioReporter” is an exogenous molecule expressed in an organism. This expression can be the result of genetic engineering, gene therapy, incorporation of a genetically altered cell or cellular product into an organism, as well as other methods known in the art for causing an organism to express an exogenous molecule or otherwise display a particular phenotype. As used herein, the term “biomarker” can encompass the term “BioReporter”, and unless otherwise specified, the characteristics described herein for biomarkers hold true for BioReporters, and vice versa.

A “BioReporter system” refers to a system for identifying a diagnostic biomarker. Such systems include an organisms expressing and/or producing a BioReporter.

As used herein, the term “organism” refers to any living entity comprised of at least one cell. A living organism can be as simple as, for example, a single eukaryotic cell or as complex as a mammal. The term “organism” encompasses naturally occurring as well as synthetic entities produced through a bioengineering method such as genetic engineering.

The term “identifying” (as in “identifying a diagnostic biomarker”) refers to methods of analyzing an object or property, and is meant to include detecting, measuring, analyzing and screening for that object or property.

The terms “nucleic acid” and “nucleotide” are used interchangeably and refer to DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole; such nucleic acids may also be referred to as bases of non-naturally occurring nucleotide mono- and higher-phosphates. Modifications can also include 3′ and 5′ modifications such as capping with a quencher, a fluorophore or another moiety.

An amino acid or nucleic acid is “homologous” to another if there is some degree of sequence identity between the two. Preferably, a homologous sequence will have at least about 85% sequence identity to the reference sequence, preferably with at least about 90% to 100% sequence identity, more preferably with at least about 91% sequence identity, with at least about 92% sequence identity, with at least about 93% sequence identity, with at least about 94% sequence identity, more preferably still with at least about 95% to 99% sequence identity, preferably with at least about 96% sequence identity, with at least about 97% sequence identity, with at least about 98% sequence identity, still more preferably with at least about 99% sequence identity, and about 100% sequence identity to the reference amino acid or nucleotide sequence.

An “isolated” molecule, such as an isolated polypeptide or isolated nucleic acid, is one which has been identified and separated and/or recovered from a component of its natural environment. The identification, separation and/or recovery are accomplished through techniques known in the art, or readily available modifications thereof.

“Polypeptide” refers to a polymer in which the monomers are amino acids and are joined together through amide bonds, alternatively referred to as a peptide. When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

As used herein, “amino acid” refers to a group of water-soluble compounds that possess both a carboxyl and an amino group attached to the same carbon atom. Amino acids can be represented by the general formula NH₂—CHR—COOH where R may be hydrogen or an organic group, which may be nonpolar, basic acidic, or polar. As used herein, “amino acid” refers to both the amino acid radical and the non-radical free amino acid.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “metabolite” is any substance produced during metabolism of another substance. A metabolite can refer to the end-product (what is remaining after metabolism) or a by-product of another compound.

The term “diagnosing disease” encompasses detecting the presence of disease, determining the risk of contracting the disease, monitoring the progress and determining the stage of the disease.

The “determining effectiveness of a treatment” includes both qualitative and quantitative analysis of effects of a treatment. Determining effectiveness of a treatment can be accomplished using in vitro and/or in vivo method. Determining effectiveness of a treatment can also be accomplished in a patient receiving the treatment or in a model system of the disease to which the treatment has been applied. In general, determining effectiveness of a treatment includes measuring a biological property at serial time points before, during and after treatment to evaluate the effects of the treatment.

“Treatment” generally refers to a therapeutic application intended to alleviate, mitigate or cure a disease or illness. Treatment may also be a therapeutic intervention meant to improve health or physiology, or to have some other effect on health, physiology and/or biological state. Treatment includes pharmacological intervention, radiation therapy, chemotherapy, transplantation of tissue (including cells, organs, and blood), and any other application intended to affect biological or pathological conditions.

The term “subject” refers to an organism that is the recipient of a biological and/or therapeutic intervention. A subject can be any organism, including cells, animals, and plants.

The term “patient” refers to a human subject that has a disease or has the potential of contracting a disease.

The term “expressing” refers to the process of creating and producing a biological feature, including genes, proteins, and physiological characteristics. Expressing a gene includes induction or production of nucleic acids encoding the gene. Expressing a protein includes translation of mRNA to produce protein encoded by a particular gene. “Expressing” also encompasses changes in configuration or structure of molecular, anatomical and cellular structures.

A “property” is any biological feature that can be detected and measured. Properties of BioReporters and biomarkers include, without limitation, expression level, pattern of expression, tissue localization and structure.

A “comparison value” results when a property at a first condition or time point is compared to the property at a second condition or time point. The comparison value can be a number or a subjective feature, such as color, structure or pattern. The comparison value may result from statistical analysis of the property at the first condition or time point and the property at a second condition or time point.

A “correlation” is determined from a comparison of comparison values. Like comparison values, correlations can be a number or a subjective feature. Correlations are generally the result of statistical analysis of comparison values. A “reference correlation” is a standard against which a correlation can be compared.

As used herein, the term “tissue” includes cells, tissues, organs, blood and plasma.

A “phenotype” is an observable physical or biochemical characteristic of an organism, as determined by both genetic makeup and environmental influences.

Introduction

The invention provides methods and compositions for identifying diagnostic biomarkers through the use of BioReporters and BioReporter systems. Diagnostic biomarkers can be indicative of a number of biological processes, including disease state, response to a therapeutic intervention (such as pharmacological treatment, radiation therapy, chemotherapy, combination therapies, and the like), and responses to physiological challenges (such as aging, environmental toxins, etc.).

In a preferred aspect, the invention provides methods and compositions for identifying diagnostic biomarkers by comparing a property of a candidate biomarker to a property of a BioReporter. BioReporters are exogenous molecules expressed in and/or produced by an organism. BioReporters are designed and selected to be associated with a certain biological or pathological state.

The “properties” of BioReporters and biomarkers used for identifying diagnostic biomarkers can be any detectable or measurable biological characteristic, including without limitation expression level, pattern of expression, tissue localization, and structure. In determining whether a candidate biomarker is a diagnostic biomarker, a property of a BioReporter is compared at a first time point and a second time point to determine a comparison value for that BioReporter. Similarly, a property of a candidate biomarker is compared at a first time point and a second time point to determine a comparison value for that candidate biomarker. The property that is analyzed for the BioReporter can be the same as or different from the property analyzed for the candidate biomarker. A comparison of the comparison values of the BioReporter and the candidate biomarker is used to determine a correlation. That correlation is in turn compared to a reference correlation, and this comparison to the reference correlation is used to determine whether the candidate biomarker is a diagnostic biomarker. Determining comparison values and correlations can be accomplished using data from in vitro or in vivo systems. Comparison values and correlations can be numerical values or subjective features such as pattern of expression. In a preferred embodiment, statistical tools are utilized to calculate the comparison values and the correlations used to identify diagnostic biomarkers.

BioReporters

BioReporters are exogenous molecules expressed in or produced by an organism. The organism expresses or produces the BioReporter as a result of genetic engineering, gene therapy, incorporation of a genetically altered cell or cellular product into an organism, xenograft of cells, tissues and organs from one organism to another, as well as by other methods known in the art for causing an organism to express and/or produce an exogenous molecule or biological characteristic.

BioReporters encompass any molecule or biological feature that can be manipulated, induced, detected, and/or quantified. In a preferred aspect, BioReporters are proteins, carbohydrates, nucleic acids, lipids, metabolites, carbohydrates, salts, and small molecules. BioReporters may also include other biological features, such as anatomical characteristics (for example, organ structure, shape and condition), cellular components (such as mitochondria and chloroplasts), and physiological features (such as blood pressure, heart rate, and respiratory rate).

In a preferred embodiment, BioReporters are proteins and nucleic acids which are expressed in an organism using genetic engineering techniques known by those of skill in the art, such as for example, by transfection of cells with a BioReporter gene construct, or by generation of transgenic animals whose genomes have been engineered to express a BioReporter gene functionally linked to a control region.

BioReporters of the invention encompass known biomarkers as well as newly generated and spontaneously occurring biomarkers which have been identified and analyzed using methods of the present invention. In a preferred embodiment, BioReporters of the invention produce a detectable signal, such as a secreted molecule or an optical signal. One example of a detectable BioReporter signal is secreted alkaline phosphatase (SEAP-Clontech). The secreted expression of SEAP is indicative of tumor burden and can be used as an evaluation tool for anticancer drug efficacy. In another exemplary embodiment, the BioReporter is firefly luciferase, which creates a luminescent signal upon application of luciferin to the organism expressing the luciferase gene.

In a preferred aspect of the invention, BioReporters possess properties which are detectable and/or quantifiable. The properties exhibited by a BioReporter will depend on the type of BioReporter. For example, BioReporters which are molecules such as proteins and nucleic acids will have properties that include expression level, patterns of expression, localization to particular tissues, and ability to bind to or be bound by substrates. BioReporters may also have properties which include anatomical characteristics, cellular shape and structure, intracellular structures, and physiological features such as blood pressure, skin color, respiratory rate, heart rate, and blood oxygen level.

In accordance with the invention, specific properties of BioReporters are associated with specific aspects of a disease or biological state. For example, the property of expression level for a particular BioReporter can be associated with the terminal stage of a cancer. In such a case, a change in expression level of the BioReporter over a period of time will indicate that the organism has reached the terminal stage of the disease. This change in expression level can also be used as a comparison value which can then be compared to properties of candidate biomarkers to determine whether those candidate biomarkers are diagnostic biomarkers that indicate that the organism has reached the terminal stage of the disease. In such a way, the known and/or measured properties of a BioReporter are used to identify diagnostic biomarkers.

In a particularly preferred embodiment, BioReporters are associated with a detectable signal. In one embodiment, the BioReporter itself produces a signal. For example, the BioReporter can be a fluorescent or luminescent protein, such as a fluorescent protein (e.g., green fluorescent protein (GFP) or blue fluorescent protein (BFP)) or luciferase. In another embodiment, the BioReporter induces a signal, for example by binding to a receptor which in turn activates the production of a secreted protein. The detectable signal produced by or induced by the BioReporter can be optical signals and secreted signals. Such detectable signals are also referred to herein as “BioReporter signals”.

In one embodiment, BioReporters are labeled with another molecule that produces a detectable signal. Such labeling may be achieved by covalently or non-covalently joining a moiety which directly or indirectly provides a detectable signal. BioReporters can be labeled either directly or indirectly. Possibilities for direct labeling include label groups: radiolabels such as ¹²⁵I, enzymes (U.S. Pat. No. 3,645,090) such as peroxidase and alkaline phosphatase, and fluorescent labels (U.S. Pat. No. 3,940,475) capable of monitoring the change in fluorescence intensity, wavelength shift, or fluorescence polarization. Possibilities for indirect labeling include biotinylation of one constituent followed by binding to avidin coupled to one of the above label groups. A label may be detectable by spectroscopic, photochemical, biochemical, immunochemical, electrical, optical or chemical means. Examples include, but are not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and colorimetric labels such as colloidal gold or colored glass or plastic (e.g., polystyrene, polypropylene, latex, etc.) beads.

In any specific embodiment of the invention, an exemplary BioReporter is secreted alkaline phosphatase, firefly luciferase, Gaussia luciferase, red fluorescent protein, green fluorescent protein, beta-2-microglobulin, allantoin (including allantoin produced by exogenous xanthine oxidase), sialylated neural cell adhesion molecule (NCAM), Pax7 (paired box gene 7), and beta-human chorionic goanotropin (B-HCG).

Candidate and Diagnostic Biomarkers

As with BioReporters, candidate and diagnostic biomarkers encompass any molecule or biological feature that can be detected, and/or quantified. In a preferred aspect, candidate biomarkers and diagnostic biomarkers are proteins, carbohydrates, nucleic acids, lipids, metabolites, carbohydrates, salts, and small molecules. Candidate and diagnostic biomarkers may also include other biological features, such as anatomical characteristics (for example, organ structure, shape and condition), cellular components (such as mitochondria and chloroplasts), and physiological features (such as blood pressure, heart rate, and respiratory rate).

Candidate biomarkers are biomarkers which have the potential to be diagnostic biomarkers, but have not yet been identified as valid diagnostic biomarkers by comparison and/or correlation to a BioReporter. Like BioReporters, candidate biomarkers possess properties which are detectable and/or quantifiable. The properties exhibited by a candidate biomarker will depend on what type of molecule or biological characteristic it is. For example, candidate biomarkers which are molecules such as proteins and nucleic acids will have properties that include expression level, patterns of expression, localization to particular tissues, and ability to bind to or be bound by substrates, while candidate biomarkers which are anatomical features will have properties that include shape, structure, and volume.

Properties of candidate biomarkers are detected, analyzed and/or quantified using techniques applicable to the type of candidate biomarker and the property in question. For example, a candidate biomarker which is a protein will have as one property its expression level. This property of the candidate biomarker can be detected, analyzed, and/or quantified using techniques that are well-known and well-established in the art, such as immunoassays (e.g., radioimmunoassay (RIA), enzyme-linked immunosorbentassay (ELISA), enzyme immunoassay (EIA), enzyme-multiplied immunoassay technique (EMIT), substrate-labeled fluorescent immunoassay (SLFIA), etc), SDS-PAGE or other electrophoresis techniques, mass spectrometry, and other methods known to one of skill in the art. Similarly, candidate biomarkers which are lipids will have properties that can be studied using assays known in the art, such as lipid assay systems comprising lipid recognition proteins and other lipid detection reagents. (see, e.g, U.S. Pat. No. 7,067,269).

In a preferred aspect of the invention, a property of a candidate biomarker is compared at a first time point to the property of that candidate biomarker at a second time point. Such a comparison can be used to determine a comparison value for that property of the candidate biomarker. Similarly, a property of a BioReporter (which may or may not be the same type of property as was determined for the candidate biomarker) can be compared at a first time point to that same property of the BioReporter at a second time point, and this comparison can be used to determine a comparison value for that property of the BioReporter. The comparison value of the BioReporter can then be compared to the comparison value of the candidate biomarker, and this comparison of the two comparison values can be used to determine a correlation for the BioReporter and the candidate biomarker properties. This correlation can then be compared to a reference correlation. Based on the comparison of the correlation and the reference correlation, it can be determined whether the candidate biomarker is indeed a diagnostic biomarker. Thus, a diagnostic biomarker is a candidate biomarker that has been analyzed and compared to a BioReporter, and based on that analysis and comparison identified as a diagnostic biomarker. Accordingly, a diagnostic biomarker, like a candidate biomarker and a BioReporter, possesses properties which are detectable and/or quantifiable. Once identified, a diagnostic biomarker can be used to measure the initiation, progression, severity, pathology, aggressiveness, grade, activity, disability, mortality, morbidity, disease sub-classification or other underlying feature of one or more biological processes, pathogenic processes, diseases, responses to therapeutic intervention, and other biological states.

In preferred embodiments of the invention, a precise statistical analysis of BioReporter and candidate biomarker properties provides significant advantage over traditional methods utilizing imaging techniques in identifying diagnostic biomarkers. “Semi-quantitative” techniques utilizing biomarkers such as luciferase, fluorescent proteins, thymidine kinase and other imaging biomarkers can be unreliable—for example, tissue diffuses and absorbs luminescence and fluorescence. The methods of the present invention can provide quantitative information on candidate biomarkers and diagnostic biomarkers through a comparison with BioReporters of the invention.

In a further embodiment of the invention, the method provides a validation step for confirming the identification of the candidate biomarker as a diagnostic biomarker. In such a validation step, a second property of the candidate biomarker is compared at a first time point and a second time point to determine a second comparison value for the candidate biomarker, and a second property of the BioReporter is compared at a first time point and a second time point to determine a second comparison value for the BioReporter. The second comparison value of the BioReporter is compared to the second comparison value of the BioReporter to determine a second correlation. This second correlation is compared with a reference second correlation, thus confirming the identification of the candidate biomarker as a diagnostic biomarker. Similarly, further validation steps utilizing a third, fourth, fifth, etc. property can be used to further confirm the identification of the candidate biomarker as a diagnostic biomarker.

In one embodiment of the invention, prior to determining comparison values for BioReporters and candidate biomarkers, the methods of the invention include a step of determining a property of the BioReporter and candidate biomarker. Determining a property includes identifying the property that will be the subject of study. Such identifying may involve selecting a known property of the BioReporter and the candidate biomarker, or it may involve using assays and detection methods known in the art to detect and identify specific properties. In a preferred embodiment, a property of a BioReporter is selected based on the disease or biological manipulation for which a diagnostic biomarker is being sought.

After being identified through the methods and compositions of the invention, diagnostic biomarkers may be further validated using methods known in the art. For example, diagnostic biomarkers can be further analyzed within an analytical test system with established performance characteristics and for which there is an established scientific framework or body of evidence that elucidates the physiological, toxicological, pharmacological, or clinical significance of test results. Generally, validation of a biomarker is context-specific and the criteria for validation will vary with the intended use of the biomarker. For example, diagnostic biomarkers for a disease with a known genetic factor can be further validated using assays that show correlation between a property of the diagnostic biomarker and that genetic factor, such as statistical correlation of expression levels, hybridization or some other molecular interaction between the biomarker and the genetic factor, similarity in expression patterns and tissue localization, as well as other methods, known in the art.

In another exemplary embodiment, diagnostic biomarkers are mediators of disease and can serve as therapeutic targets as well as diagnostic tools. For example, several secreted proteases activate or inactivate cytokines associated with or physically attached to a connective tissue matrix. Some studies have shown that tumor cells will not metastasize unless normal white blood cells are induced into secreting protease MMP-9, which encourages blood vessel growth through the connective tissue to the tumor. (Hanahan et al., (2006), PNAS, 103(33):12493-12498). Therapeutic treatments directed to inhibiting such a biomarker would thus serve to indirectly diminish and prevent the growth of tumors by altering the tissue microenvironment.

BioReporter Systems

A “BioReporter system” is a system utilizing BioReporters to identify diagnostic biomarkers. In general, a BioReporter system is an organism expressing a BioReporter that can be used to identify and analyze candidate and diagnostic biomarkers. BioReporter systems can include single celled organisms, cell lines, tissues, plants and animals. In a preferred embodiment, BioReporter systems are animal models which have been engineered to express specific BioReporters in diseased tissue, normal tissue, or body fluids. These BioReporters can be engineered into transgenic as well as xenograft animal models. In addition to the organism expressing the BioReporter, BioReporter systems can also include imaging equipment, molecular assays, databases and computer algorithms and systems for use with and study of BioReporter and biomarker signals.

In one embodiment, animal models used as BioReporter systems are preclinical animal models. Preclinical animal models, also referred to as animal models of disease, are non-human animals with a disease or injury that is similar to a human condition. The use of animal models allows researchers to investigate disease states in ways which would be inaccessible in a human patient. Preclinical animal models include animal models which have established markers of a particular disease and which can be used to study the pathology, the diagnosis and treatment of disease. Animal models of disease can be spontaneous (naturally occurring in animals), or be induced by physical, chemical or biological means. Examples of animal models include transgenic animals engineered to develop tumors associated with particular cancers, animals which have been induced to develop epilepsy using pharmcological agents such as metrazol (pentylenetetrazol), animals which have been immunized with an auto-antigen to induce an immune response that models autoimmune diseases, animals which have been physically altered to induce the symptoms of a particular disease state, such as by occluding the middle cerebral artery to create an animal model of ischemic stroke, animals infected with pathogens to reproduce human infectious diseases, and mice genetically altered to induce disease states for which all genetic causes are not necessarily known (such as in producing obese mice which develop Type II diabetes).

In a preferred aspect, the invention provides animal models which have been engineered to express specific BioReporters. These BioReporter animal models may or may not also exhibit properties of a particular disease or physiological state.

In an exemplary embodiment, the invention provides a BioReporter system comprising a mouse reporter strain expressing both firefly luciferase and a human placental secreted alkaline phosphatase (the “LUSAP” mouse). The LUSAP mouse facilitates dual spatial detection and quantification of cells of interest. In a preferred embodiment, the LUSAP mouse is a conditional genetic model employing Cre/LoxP technology, which is known in the art (see e.g., Lyons et al. (2003), Cancer Res, 63:7042-46; Safran et al., (2003), Mol Imaging, 2:297-302). The LUSAP mouse is a dual BioReporter system in which two detectable signals (luminescence and secreted alkaline phosphatase) can be monitored. These BioReporter signals are then used in accordance with the invention to identify diagnostic biomarkers. In a further embodiment, the LUSAP mouse is also an animal model of disease, and its BioReporter signals are associated with particular aspects of the disease.

In another exemplary embodiment, a transgenic mouse is provided which expresses the BIoReporter beta-2 microglobulin (B2M mouse). This BioReporter mouse system secretes human beta-2-microglobulin into the serum and the urine. This BioReporter can be detected by antigenicity using techniques described herein and known in the art.

In still another exemplary embodiment, the BioReporter mouse system produces, extra urate oxidase (UOX mouse) and has an increased capacity to convert uric acid to allantoin. Allantoin can be detected in the urine or serum. This embodiment of the BioReporter system is particularly amenable for use in animal models of cancer, because tumors make more uric acid than normal cells, and this BioReporter creates more allantoin than is normally produced, thus further increasing the signal-to-noise ratio of the BioReporter and in turn the sensitivity of tests utilizing this BioReporter.

BioReporter systems are created according to the present invention using techniques known in the art. For example, to create BioReporter systems made up of cells and cell lines, methods such as transfection, electroporation, microinjection, infection with a viral or bacteriophage vector containing the nucleic acid sequences, cell fusion, chromosome-mediated gene transfer, microcell-mediated gene transfer, spheroplast fusion, and the like may be used. Numerous techniques are known in the art for the introduction of foreign genes into cells (see, e.g., Loeffler and Behr, Meth. Enzymol. 217:599 618 (1993); Cohen et al., Meth. Enzymol. 217:618 644 (1993) which are hereby incorporated by reference).

Similarly, transgenic plants and animals can be created to express and/or produce BioReporters by incorporating genes encoding the BioReporter of interest into the genomes of these organisms. Transgenic mice are achieved routinely in the art using the technique of microinjection, as described in U.S. Pat. No. 4,736,866 and by B. Hogan et al. in “Manipulating the Mouse Embryo: A Laboratory Manual”, Ed. 2, pp. 89 204. Plainview, N.Y.: Cold Spring Harbor Laboratory, USA (1995). Further methods for the production of a transgenic non-human animal, for example a transgenic mouse, comprise introduction of a targeting vector into a germ cell, an embryonic cell, stem cell or an egg or a cell derived therefrom.

In a particularly preferred embodiment, the invention provides BioReporter systems that include Cre-inducible reporter mouse lines, which can be used for identifying cells of a specific lineage as well as all of the cells that are derived from the cells that were originally (genetically) marked. The DNA recombinase Cre can permanently rearrange genomic DNA where short 34 base pair LoxP sites have been transgenically engineered into mouse loci. In conditional mouse lines, Cre can mediate the inactivation of genes (i.e., conditional knockout alleles) or the activation of genes (i.e., conditional knock-in alleles & conditional reporter alleles). Such mouse lines can be established using methods known in the art. (see e.g., Brocard et al., (1997) PNAS, 94:14559-14563; Lyons et al., (2003), Cancer Res, 63:7042-7046, Vasioukhin et al., (1999), PNAS, 96:8551-8556, which are hereby incorporated by reference in their entirety).

Detection and Analysis of BioReporters and Biomarkers

In a preferred aspect, the invention provides methods and compositions for identifying diagnostic biomarkers by comparison of properties of candidate biomarkers to properties of BioReporters.

BioReporters and biomarkers analyzed and detected according to methods of the present invention can be studied in vivo or in vitro. In a preferred embodiment, detection and analysis of BioReporters and biomarkers are conducted on samples from an organism. The samples used in these methods can include plasma, biological fluids and cells, and mixtures thereof.

In one aspect of the invention, a property of a candidate biomarker is compared at a first time point to the property of that candidate biomarker at a second time point. Such a comparison is used to determine a comparison value for that property of the candidate biomarker. Similarly, a property of a BioReporter (which may or may not be the same type of property as was determined for the candidate biomarker) is compared at a first time point to that same property of the BioReporter at a second time point, and this comparison is used to determine a comparison value for that property of the BioReporter. The comparison value of the BioReporter can then be compared to the comparison value of the candidate biomarker, and this comparison of the two comparison values can be used to determine a correlation for the BioReporter and the candidate biomarker properties. This correlation can then be compared to a reference correlation. Based on the comparison of the correlation and the reference correlation, it can be determined whether the candidate biomarker is indeed a diagnostic biomarker.

In an exemplary embodiment, a BioReporter is a protein whose expression increases when a disease is contracted by an organism. In accordance with the invention, the expression level of the BioReporter is compared at a first time point (i.e., before the organism contracts the disease) to the expression level of the BioReporter at a second time point (i.e., after the organism contracts the disease). This comparison is used to determine a comparison value for the expression level of the BioReporter. Similarly, a property of a candidate biomarker is compared at a first time point to that property of the candidate biomarker at a second time point to determine a comparison value for the property of the candidate biomarker. The comparison value of the property of the candidate biomarker can then be compared to the comparison value of the expression level of the BioReporter to determine a correlation. This correlation is then compared to a reference correlation, and this comparison is used to determine whether the candidate biomarker is a diagnostic biomarker. The property of the candidate biomarker that is correlated to the BioReporter's expression level does not necessarily have to be the same property, i.e., the property of the candidate biomarker can be expression level but does not necessarily have to be. For example, another property of the candidate biomarker, such as its structure, can be compared to the BioReporter's expression level. If a change in the candidate biomarker's structure occurs when the change in the BioReporter's expression level occurs, then those two properties can be seen as correlated, thus identifying the candidate biomarker as a diagnostic biomarker.

The time points at which a property of a candidate biomarker are measured to determine a comparison value for that candidate biomarker may or may not be the same time points used to determine a comparison value for a BioReporter. In addition, multiple time points may be used to determine comparison values for both the candidate biomarker and the BioReporter, and different combinations of those time points may also be used to determine comparison values. For example, a property of a candidate biomarker at a first time point can be compared to a property of that candidate biomarker at a third time point, and this comparison can then be used to determine a comparison value for that candidate biomarker. Similarly, a property at a second time point can be compared to a property at a third, fourth, fifth, etc. time point to determine a comparison value. In another example, a comparison value is determined from a comparison of a property at more than two time points.

As described herein, the comparison value of a BioReporter is compared to a comparison value of a candidate biomarker to determine a correlation. This correlation is then compared to a reference correlation to identify the candidate biomarker as a diagnostic biomarker. In one embodiment, the reference correlation is a threshold value. This threshold value may be a numerical quantity or a pattern (such as a pattern of expression). A correlation determined from a comparison of a comparison value for a candidate biomarker to a comparison value of a BioReporter can be compared to such a reference correlation, and a difference between the correlation and the reference correlation identifies the candidate biomarker as a diagnostic biomarker. The difference between the correlation and the threshold value of the reference correlation includes the correlation having a value greater than the threshold value or less than the threshold value. If the threshold value is a pattern, then a difference between the pattern of the correlation and the threshold value identifies the candidate biomarker and the diagnostic biomarker.

A biomarker is considered to be informative if a measurable aspect of the biomarker is associated with a given phenotype, such as a particular disease state in an organism. Such a measurable aspect may include, for example, the presence, absence, or concentration of the biomarker in the biological sample from the individual and/or its presence as part of a profile of biomarkers. Such a measurable aspect is also described herein as a property. A property of a biomarker may also be a ratio of two or more measurable aspects of biomarkers, which biomarkers may or may not be of known identity. A “biomarker profile” comprises at least two such properties, where the properties can correspond to the same or different classes of biomarkers such as, for example, a nucleic acid and a carbohydrate. A biomarker profile may also comprise at least three, four, five, 10, 20, 30 or more properties. In one embodiment, a biomarker profile comprises hundreds, or even thousands, of features. In another embodiment, the biomarker profile comprises at least one measurable aspect of at least one internal standard. Biomarker profiles of candidate biomarker and of BioReporters can be used in accordance with the invention to identify diagnostic biomarkers.

As discussed herein, the properties of the biomarkers and the BioReporters analyzed according to the invention can be any biological, chemical, or physical attribute of the BioReporter or biomarker that can be detected and/or measured. For example, if a biomarker and a BioReporter are proteins, properties of such proteins that could be used according to the invention include expression level, expression pattern, tissue localization, antigenicity, ability to bind to a substrate, primary, secondary and tertiary structure, as well as other aspects well known in the art.

The present invention is not limited in the type, characteristic, or form of the methods used to detect and analyze properties of BioReporters and biomarkers, and the method chosen to detect and analyze a particular property will depend on the type of BioReporter/biomarker and the property being studied. For example, if the biomarker is a nucleic acid and the property being analyzed is expression level, then methods of detection and analysis can utilize (without limitation) microarrays, polymerase chain reaction (PCR), electrophoresis, Northern or Southern blots, and spectroscopy. Such techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference). Similarly, other kinds of biomarkers and BioReporters can be detected and analyzed using appropriate methods known in the art. For example, colorimetric assays using dyes are widely available. Alternatively, detection may be accomplished spectroscopically. Spectroscopic detectors rely on a change in refractive index; ultraviolet and/or visible light absorption, or fluorescence after excitation with a suitable wavelength to detect reaction components. Exemplary detection methods include fluorimetry, absorbance, reflectance, and transmittance spectroscopy. Changes in birefringence, refractive index, or diffraction may also be used to monitor complex formation or reaction progression. Particularly useful techniques for detecting molecular interactions include surface plasmon resonance, ellipsometry, resonant mirror techniques, grating-coupled waveguide techniques, and multi-polar resonance spectroscopy. These techniques and others are well known and can readily be applied to the present invention by one skilled in the art, without undue experimentation.

For protein biomarkers, a preferred method of detection is mass spectroscopy. Mass spectroscopy techniques include, but are not limited to ionization (I) techniques such as matrix assisted laser desorption (MALDI), continuous or pulsed electrospray (ESI) and related methods (e.g., IONSPRAY or THERMOSPRAY), or massive cluster impact (MCI); these ion sources can be matched with detection formats including linear or non-linear reflection time-of-flight (TOF), single or multiple quadropole, single or multiple magnetic sector, Fourier Transform ion cyclotron resonance (FTICR), ion trap, and combinations thereof (e.g., ion-trap/time-of-flight). For ionization, numerous matrix/wavelength combinations (MALDI) or solvent combinations (ESI) can be employed. Subattomole levels of analyte have been detected, for example, using ESI (Valaskovic, G. A. et al., (1996) Science 273:1199-1202) or MALDI (Li, L. et al., (1996) J. Am. Chem. Soc. 118:1662-1663) mass spectrometry. ES mass spectrometry has been introduced by Fenn et al. (J. Phys. Chem. 88, 4451-59 (1984); PCT Application No. WO 90/14148) and current applications are summarized in review articles (R. D. Smith et al., Anal. Chem. 62, 882-89 (1990) and B. Ardrey, Electrospray Mass Spectrometry, Spectroscopy Europe, 4, 10-18 (1992)). MALDI-TOF mass spectrometry has been introduced by Hillenkamp et al. (“Matrix Assisted UV-Laser Desorption/Ionization: A New Approach to Mass Spectrometry of Large Biomolecules,” Biological Mass Spectrometry (Burlingame and McCloskey, editors), Elsevier Science Publishers, Amsterdam, pp. 49-60, 1990).

Another method of detection widely used is electrophoresis separation based on one or more physical properties of the biomarker/BioReporter of interest. A particularly preferred embodiment for analysis of polypeptide and protein biomarkers is two-dimensional electrophoresis. A preferred application separates the analyte by iso-electric point in the first dimension, and by size in the second dimension. Methods for electrophoretic analysis of biomarkers will vary widely with the biomarker being studied, but techniques for identifying a particular electrophoretic method suitable for a given molecule are well known to those of skill in the art.

Detection devices can comprise any device or use any technique that is able to detect the presence and/or level of a biomarker or BioReporter in a sample. Examples of detection techniques that can be used in a detection device include, but are not limited to, nuclear magnetic resonance (NMR) spectroscopy, 2-D PAGE technology, Western blot technology, immunoanalysis technology, electrochemical detectors, spectroscopic detectors, luminescent detectors, and mass spectrometry. The output from a detection device can be processed, stored, and further analyzed or assayed using a bio-informatics system. A bio-informatics system can include one or more of the following: a computer; a plurality of computers connected to a network; a signal processing tool(s); and a pattern recognition tool(s).

Any disease or biological state with detectable biological characteristics can be analyzed using methods of the invention. Diseases for which the methods and compositions of the invention are applicable include without limitation sarcopenia, cancer, neurodegenerative disease, diabetes, and cardiovascular disease. Biological states for which the methods and compositions of the invention are applicable includes without limitation the response of an organism to a treatment (including pharmacological treatment, radiation therapy, chemotherapy, surgery, organ transplant, and other therapeutic interventions).

Correlation of BioReporters and Candidate Biomarkers

In a preferred aspect, a comparison value for a candidate biomarker is compared to a comparison value for a BioReporter to determine a correlation for the comparison value of the candidate biomarker and the comparison value of the BioReporter. In one embodiment, this correlation is generated using statistical techniques known in the art. In another embodiment, this correlation is a comparison of pattern or of some other subjective feature.

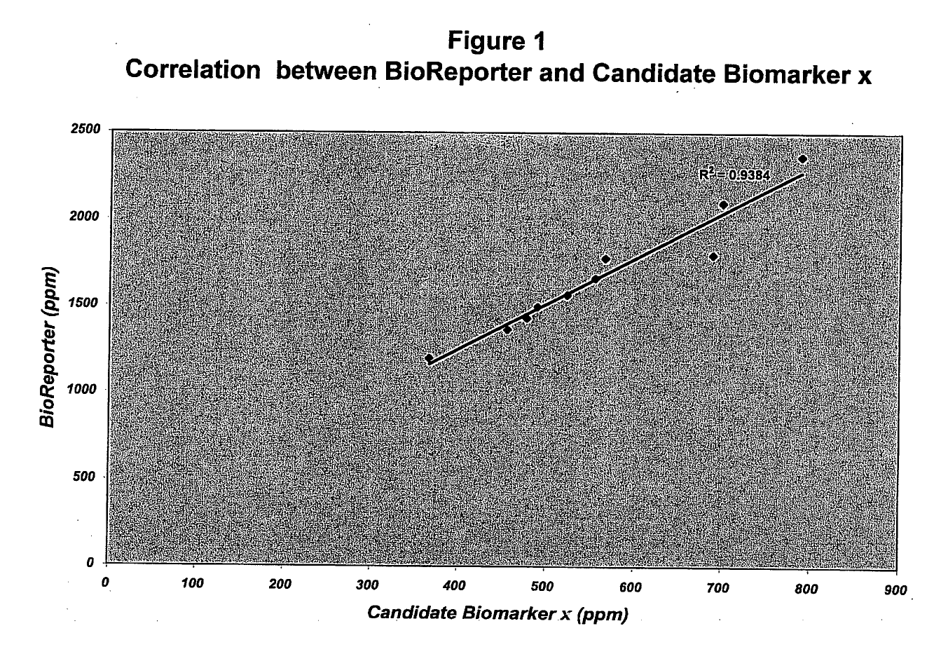

In an exemplary embodiment, a series of expression data set of a BioReporter taken at various times, doses, or longitudinal disease stages is obtained and plotted against a corresponding series of expression data set of a candidate biomarker concurrently. This plot is used to determine a correlation for the candidate biomarker and the BioReporter. As +illustrated in FIG. 1, the level of correlation between the two data sets can be measured by tools such as squared errors (R 2). Generally, the closer to 1 of the value of R², the more correlated the two data sets are. Having a value of 1 is idea, but generally, even for the correlation between two duplicate data sets from one same sample, a R² value of 0.9 to 0.95 is statistically satisfactory.

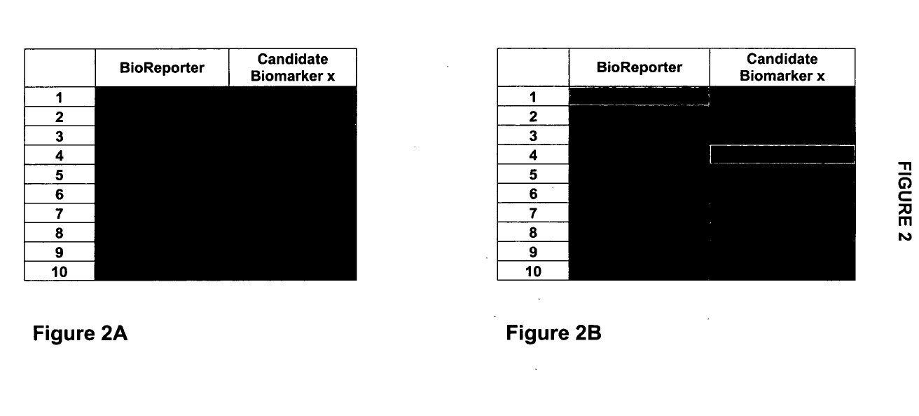

In another exemplary embodiment, the level of correlation between one data set of a candidate biomarker and another data set of a BioReporter is obtained using a fold change color visualization system. The expression of a BioReporter at one condition or time point is compared to that at a control point (such as a time-point before any drug administration is initiated). The resulted ratio of expression is the fold change for that comparison. There is a fold change value for each experimental condition tested in relation to a common control. As a result, a series of fold change data set is obtained for both the BioReporter and any candidate biomarker. A distinctive color is assigned to each range of fold changes. For example, one may assign a bright red for fold changes at least 2 or above; likewise, a deep blue is chosen to represent fold changes 0.5 or less (in another word: a down-regulation of at least 2 fold or more). Color designation is purely arbitrary and subject to a user's personal preference. Once the color designation is done, a user is able to determine the level of correlation based on level of color consistency. As illustrated in FIG. 2A, the expression fold changes for both the BioReporter and the Candidate biomarker x are shown both red at condition point #1, #2, #4, #7, and both blue at condition point #3, #6, #8, #9, but inconsistent at condition point #5, #10. By this visual assessment, a researcher is able to say the BioReporter and the candidate biomarker x respond similarly to eight of the ten experimental conditions. Alternatively, the color scheme is further detailed to differentiate the degree of fold changes. For example as illustrated in FIG. 2B, a color pink represents a stimulatory response with fold change from 1.5 to 2; a bright red remains representative of a stimulatory fold change of 2 and up. Likewise, a light blue shows an inhibitory response with fold change from 1.5 to 2; a deep blue indicates an inhibitory response with fold change of at least 2 and up. When coupled with the application of the squared errors approach, this more detailed color scheme assessment offers a researcher more flexibility to categorize physiological, pathological, and pharmacological changes happening in a living organism.

Additionally, other statistical tools are applicable to determining comparison values and correlations. These tools include supervised or unsupervised classification models, multidimensional profile classification, linear discrimination and/or support vector machines, and boosted logistic regression. In addition, some well known statistical tests and procedures for research observations are: Student's t-test, chi-square test, analysis of variance (ANOVA), Mann-Whitney U, Regression analysis, factor analysis, statistical correlation, Pearson product-moment correlation coefficient, and Spearman's rank correlation coefficient. Any of these statistical tools, as well as others known in the art, can be used to determine comparison values, to determine correlations, and to compare a correlation to a reference correlation.

Methods for manipulating and analyzing data to detect and analyze patterns are known in the art, and such methods are applicable to the determination of comparison values and correlations described herein. For example, comparison values and correlation can be determined using known pattern recognition methods and comparisons of frequencies of occurrence of properties. (see, e.g, Wang et al., eds., Pattern discovery in Biomolecular Data: Tools, Techniques, and Applications, (1999); Andrews, Introduction to mathematical techniques in pattern recognition; (1972); Fu et al., eds., Applications of Pattern Recognition, (1982); Pal et al., eds., Genetic Algorithms for Pattern Recognition, (1996); Chen et al., eds., Handbook of pattern recognition & computer vision (1999); Friedman, Introduction to Pattern Recognition Statistical, Structural, Neural, and Fuzzy Logic Approaches, (1999) all of which are expressly incorporated by reference.) Such methods can be used with more “objective” data that lead to numerical values as well as with “subjective” data, such as expression patterns, color (of hair, eyes, skin), and tissue localization.

Uses for Diagnostic Biomarkers

In a preferred aspect, BioReporters and BioReporter systems are used to identify diagnostic biomarkers. The identification of diagnostic biomarkers is applicable to various aspects of biomedical research, including every phase of drug development, from drug discovery and preclinical evaluations through each phase of clinical trials and into post-marketing studies. Diagnostic biomarkers can be used to predict a patient's response to a compound, act as a surrogate endpoint, and aid in making efficacious and cost-saving decisions or terminating drug entities more quickly during the research process. Patient enrichment strategies can also utilize diagnostic biomarkers to identify certain patient populations that are more likely to respond to the drug therapy or to avoid specific adverse events.

Diagnostic biomarkers can also be used in diagnosing disease. As used herein, the term “diagnosing disease” includes detecting the presence of a disease, determining risk of contracting a disease, determining the extent and or stage of a disease, determining a prognosis for survival, and monitoring progression of a disease over time. Diagnostic biomarkers can be used to detect and analyze all of these different aspects of diagnosing disease.

Diagnostic biomarkers can also be used to study and monitor the effect of a treatment protocol. As discussed above for diagnosing a disease, a diagnostic biomarker that has a measurable property that changes in response to a treatment protocol can be used to identify diagnostic biomarkers that can also provide information regarding the effects of that treatment protocol.

Diagnostic biomarkers of the invention can also be incorporated into kits, which are then used for various research and clinical applications, including diagnosing disease and determining the effectiveness of a treatment. In a preferred embodiment, such kits include an isolated diagnostic biomarker, a container that includes the isolated diagnostic biomarker, and instructions for using the kit. In a preferred embodiment, the isolated diagnostic biomarker included in kits of the invention will be identified using the methods and compositions described herein. In a further embodiment, the instructions included in kits of the invention will provide methods for using the kits to diagnose disease, determine the effectiveness of a treatment, and identify causative factors of a disease or other biological condition. In a further embodiment, the containers of the kits of the invention include the isolated biomarker in a standardized solution or immobilized on a substrate.

Diagnostic biomarkers can also be used to develop libraries of biomarkers. In a preferred embodiment, libraries of diagnostic biomarkers comprise more than 10 biomarkers, preferably more than 100 biomarkers, and more preferably more than 1000 biomarkers. Libraries of biomarkers according to the invention include biomarkers in solution, biomarkers immobilized on a substrate, as well as digital information related to biomarkers (stored in a user accessible medium such as a computer), such as nucleic acid sequence and structure, biomarker amino acid sequence and structure, pattern of expression, tissue localization, imaging data of optical signals generated by biomarkers in an organism, and other types of information related to biomarker properties that can be stored in a digital format. Libraries of diagnostic biomarkers can thus include organisms as well as digital data.

The present invention may be better understood by reference to the following non-limiting Examples, which are provided as exemplary of the invention. The following examples are presented in order to more fully illustrate preferred embodiments of the invention, but should in no way be construed as limiting the broad scope of the invention.

While this invention has been disclosed with reference to specific embodiments, it is apparent that other embodiments and variations of this invention may be devised by others skilled in the art without departing from the true spirit and scope of the invention.

All patents, patent applications, and other publications cited in this application are incorporated by reference in the entirety.

### EXAMPLES

### Example 1

### Generation of Dual Reporter Mouse Line

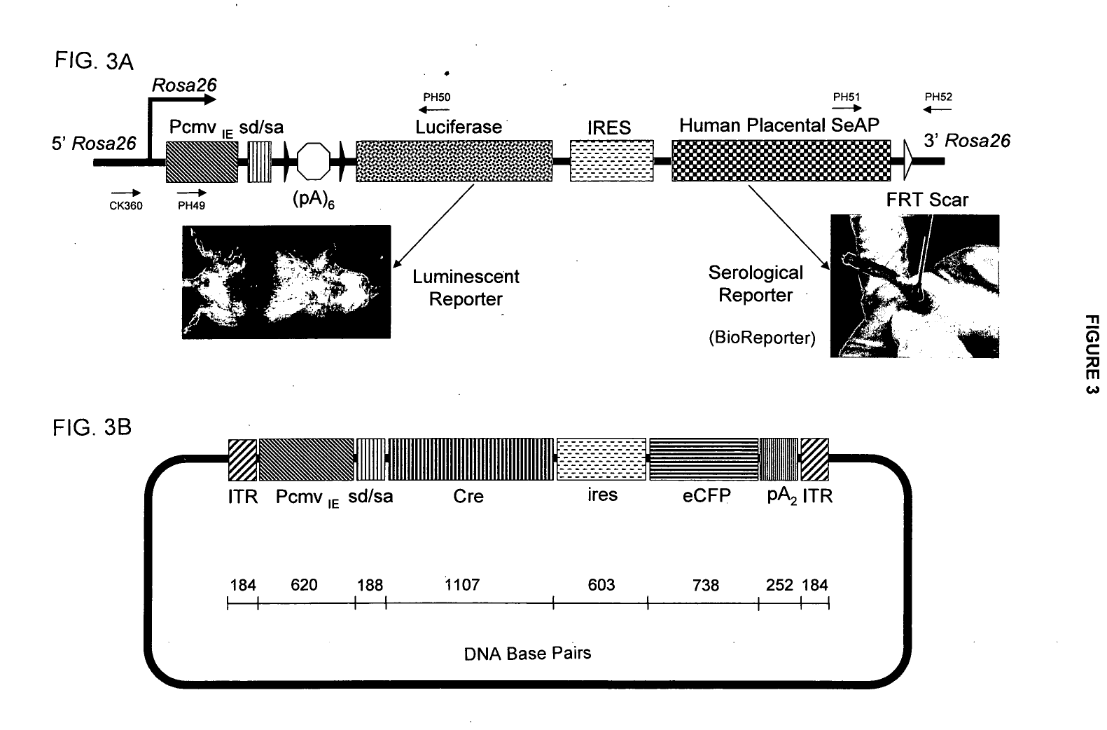

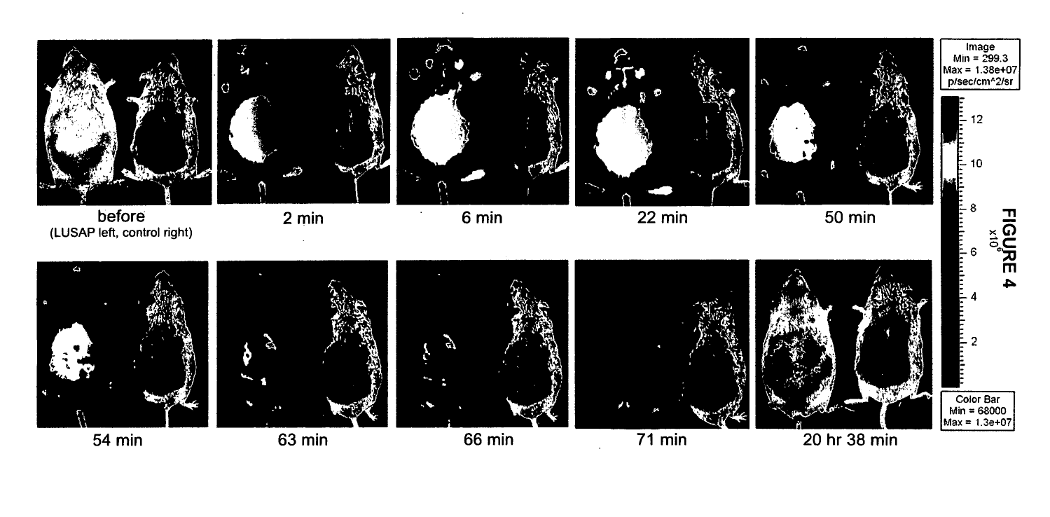

The conditional LUSAP reporter allele was designed to express both firefly luciferase and human placental secreted alkaline phosphatase constitutively at high levels following activation by Cre recombinase (FIG. 3A). The two reporter genes were expressed as tandem cistron by means of a human internal ribosome site (IRES). The construct was targeted to the Rosa26 locus in a manner similar to methods known in the art (see, e.g., Safran et al., (2003), Mol Imaging, 2:297-302; Soriano, (1999), Nat Gen 21:70-71; Srinivas, et al., (2001), BMC Dev Biol, 1:4) except that the native Rosa26 promoter was augmented by a CMV immediate-early promoter/enhancer and SV40 late viral protein gene 16S/19S splice donor and acceptor signal sites to maximize ubiquitous expression. A pRosa26-1 plasmid (Soriano, (1999), Nat Gen 21:70-71) containing genomic DNA for the Rosa26 locus was used. A targeting vector was constructed which consisted of (in 5′ to 3′ order): the 1.1 kB of 5′ Rosa26 homology, the CMV immediate-early promoter/enhancer and the SV40 late viral protein gene 16S/19S splice donor and acceptor signal sites, a stop cassette consisting of six copies of the SV40 viral early and late polyadenylation signal flanked by LoxP sites, the firefly luciferase gene (pGL3, Promega, Madison, Wis.), a human internal ribosome entry site (IRES) from the NF-kB repressing factor, and the human placental secreted alkaline phosphatase gene (pSEAP2-Basic, Clontech, Mountain View, Calif.), an FRT-flanked neomycin resistance gene (Neo), the 4.2 kB of 3′ Rosa26 homology, and the PYF enhancer driving the thymidine kinase gene.

Germline mice were established carrying the Neo-containing allele (Rosa26^{LUSAPp/WT}). After removing the Neo positive selection cassette by Flpe-mediated recombination, Neo excised Rosa26^{LUSAPm/WT} mice proved to be viable and fertile as heterozygotes or homozygotes.

For focal Cre activation, a Type 2 adeno-associated virus plasmid was constructed with Cre expressed from a promoter known to be efficiently expressed in skeletal muscle (FIG. 3B). As an internal control for AAV infection, the enhanced cyan fluorescent protein (eCFP) was designed to be expressed from the construct as a second cistron by means of an encephalomyocarditis virus ires. Efficient packaging of the plasmid was achieved to a titer of 4.4×10¹³ particles/ml.

The linearized targeting vector was electroporated into R1 mouse embryonic stem cells using techniques known in the art (see e.g, Nagy et al., (2002), PNAS, 90:8424-8428), and the cells were subjected to positive and negative selection. A correctly targeted clones was identified by a downshift from 11 kB to 9 kB by Southern hybridization using a 5′ external probe and digestion by EcoRV. The EcoRV site within the construct was contained in the FRT-flanked Neo cassette.

Microdeletion was ruled out by Southern hybridization with an internal probe to Neo using EcoRV, BamHI, or SalI digestions. Cells from this ES cell clone were microinjected into C57BL/6 blastocysts in order to generate chimeric mice. Chimeric mice were mated to C57BL/6 dams, and their agouti offspring were confirmed to harbor the targeted allele by Southern hybridization. Germline mice were designated to have the genotype, Rosa26^{LUSAPp/WT}. The FRT-flanked Neo cassette was removed by breeding Rosa26^{LUSAPp/WT} mice to transgenic mice expressing Flp-e, thereby generating Rosa26^{LUSAPm/WT} mice (i.e., Neo minus).

For genotyping the LUSAP mouse line, the 5′ and 3′ primers were (ck360, 5′-AAAGTCGCTCTGAGTTGTTATCA-3′; ph49, 5′-CCGCCAGATTCTGACATGGA-3′; ph51, 5′-GCGCACCCGGGTTACTCTA-3′) and (ph50, 5′-TTCCAGGAACCAGGGCGTAT-3′; ph52, 5′-CAGAAGACTCCCGCCCATCT-3′; ba97, 5′-GATCTGGACGAAGAGCATCA-3′), respectively. The primer ba97 is only necessary for detection of the Rosa^{26LUSAPp} allele. DNA was extracted from tails and 2 μl (5-20 ng) was used in the subsequent PCR reaction. Each 25 μl PCR reaction contained 1× buffer, 2 mM MgCl₂, 200 uM deoxynucleotides, 0.2 μM primers, and 0.4 U of Taq DNA polymerase (Promega). Cycling conditions were: 95° C. for 5 minutes, 32 cycles of 95° C. for 30 seconds/64° C. for 20 seconds/72° C. for 120 seconds, followed by 72° C. for 7 minutes. The wild type, LUSAPp (Neo plus), or LUSAPm (Neo minus), and GoLUSAP (Cre-activated) alleles resulted in 238, 668, 354, or 391 bp bands, respectively.

### Example 2

### Optical Imaging of BioReporter Mice

Serial luminescence imaging of LUSAP BioReporter mice was performed before and after intraperitoneal (IP) injection with a single luciferin dose, 150 mg/kg (10 μl/g) body weight (Caliper—Xenogen, Alameda, Calif.). For firefly luciferase imaging of the right thigh, a mouse expressing luciferase as a Cre/LoxP reporter allele was intramuscularly administered with 0.1 ml (5×10¹⁰ particles) of an adenoassociated virus constitutively expressing Cre (AAVcre). After three weeks of AAVcre injection the mouse was administered with an intraperitoneal injection of a single luciferin dose, 10 μl/g body weight.

A Z/RED-Tg^{GoZRED/WT} HPRT^{Cre/WT} monomeric red fluorescent protein reporter mouse with ubiquitous Cre activation was shaved in the belly region before imaging. Luminescent and fluorescent imaging of live animals was performed using Xenogen IVIS® 200 system (Caliper—Xenogen). The Xenogen instrument employs a scientific grade, cryogenically cooled CCD camera which has a low-noise, 16 bit digitized electronic readout. The animals were maintained under inhaled anesthesia using 2% isoflurane in 100% oxygen at the rate of 2.5 liters per minute.

For firefly luciferase imaging, the image acquisition parameters were 50 sec exposure time, 2×2 binning, 12.6 cm field of view, and f/stop of 1/4. For the luminescent AAVcre experiment, the imaging parameters of 60 sec exposure time, 2×2 binning, 12.6 cm field of view, and f/stop of ¼ were used.

For fluorescent Z/RED mouse imaging, acquisition was accomplished using excitation and emission filters for DsRED, 2×2 binning, 0.5 sec exposure time and f/stop of 8/4. Luminescent and fluorescent data was acquired and analyzed using the manufacturer's proprietary Living Image 2.5© software.

### Example 3

### Serological Bioassay Measurement

For detection of SEAP in the bloodstream, a blood sample was isolated from the animal through saphenous vein puncture into a microfuge tube with minimal hemolysis. The blood was allowed to clot at RT for 30-60 minutes (min) and was centrifuged at 2500×g for 15 min at 4° C. The clear/yellow supernatant serum was removed to a fresh tube and stored at −20° C. or assayed immediately with the BD Great EscAPe™ SEAP chemiluminescent assay (BD Biosciences Clontech, Palo Alto, Calif.) according to the manufacturer's instructions, which includes a 30 minute 65° C. heating step to inactivate endogenous murine serum phosphatases. Assay samples containing 12.5 ul serum each were measured for luminescent signal using Xenogen IVIS® 200 system (Caliper—Xenogen). Imaging was performed 15 min after sample preparation with the standard settings of 60 sec exposure time, 2×2 binning, 12.6 cm field of view, and f/stop of 2/4.

### Example 4

### Expression of Luciferase Upon Activation of the Reporter Allele

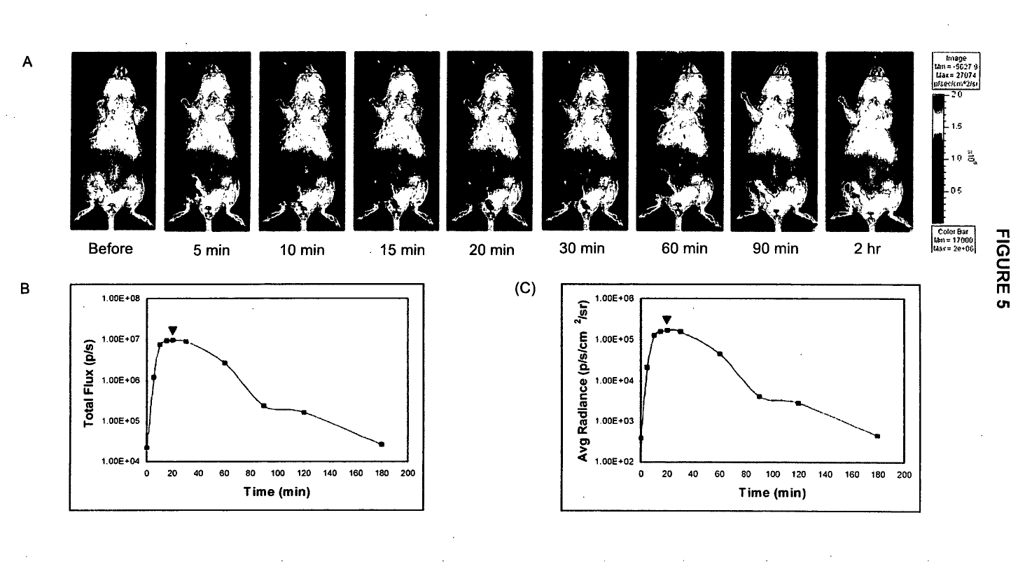

To determine the intensity and time course of luciferase expression, reporter mice carrying the Rosa26^{LUSAPm/WT} allele were bred to HPRT^{Cre/WT} mice expressing Cre ubiquitously. A 9 month old double heterozygote Rosa26^{GoLUSAP/WT} HPR^{Cre/WT} mouse and wild type control were injected with a single dose of luciferin. Overall luciferase signal was maximal at 22 minutes after luciferin injection, but intraperitoneal luciferase signal and hematogenous luciferase signal (seen in the hairless paws and tail base) persisted for more than 20 hours (FIG. 4). Maximum signal intensity from the luciferase in our bi-cistronic reporter was 1.3×10₇ photons/cm₂/sec/steradian which is comparable to or greater than established, useful monocistronic luciferase reporters known in the art (see, e.g., Lyons et al. (2003), Cancer Res, 63:7042-46; Safran et al., (2003), Mol Imaging, 2:297-302; and Uhrbom et al., (2004), Nat Med, 10: 1257-1260).

To determine the optimal interval after luciferin injection for imaging extraperitoneal luminescence, the right thigh of a Rosa26^{LUSAPm/WT} reporter mouse was injected with 5×10¹⁰ particles of packaged AAVcre. Three weeks later, the mouse was injected with luciferin and serially imaged for 2 hours (FIG. 5A). Focal luciferase signal was maximal at 20 minutes with a total flux of 1.54×10⁹ photons/sec and a normalized average radiance of 2.73×10⁶ photons/cm²/sec/steradian (FIGS. 5B and 5C). However, a window of nearly equivalent signal occurred between 15 and 30 minutes following luciferin injection.

### Example 5

### Analysis of Signal Intensity of BioReporters

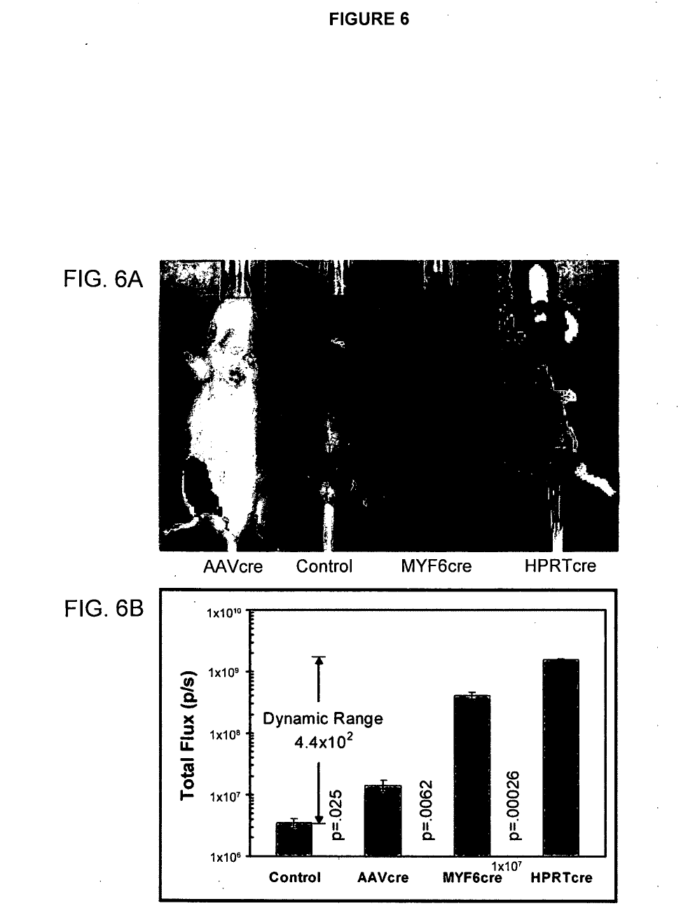

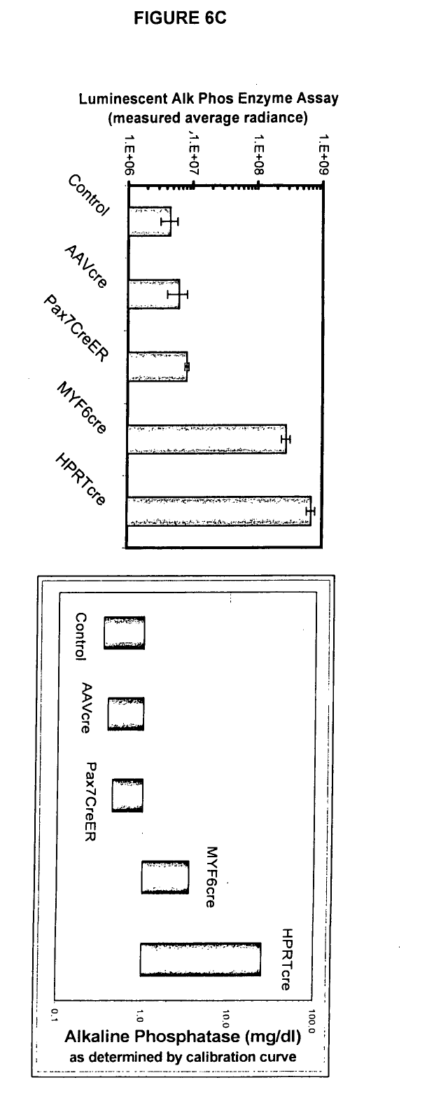

Mice were generated to demonstrate the range of signal intensity for focal, lineage-restricted, and ubiquitous reporter activity of the luciferase biomarker and the serum SeAP biomarker. For focal activation we utilized a 12 month old Rosa26^{LUSAPm/WT} AAVcre mouse (FIG. 5), whereas for activation of the biomarkers in the maturing skeletal muscle lineage we utilized a 9 week old Rosa26^{LUSAPm/WT} Myf6^{ICNm/WT} mouse. For ubiquitous activation, we employed a 7 week old Rosa26^{GoLUSAP/WT} HPRT^{Cre/WT} mouse and a 9 week old Rosa26^{LUSAPm/WT} littermate control that did not carry Cre. Luciferase signal is shown in FIG. 6A.

Quantification of luciferase signal (FIG. 6B) revealed a substantial 4.4×10² photons/cm₂/sec/steradian range of signal above background for ubiquitous activation versus wild type control, with statistically-significant differences between control, focal, lineage restricted, and ubiquitous activation of the reporter (t-test between groups, p<0.025). The calibration curve for the SeAP assay, determined by serial dilution of serum from a Rosa26^{LUSAPm/WT} Myf6^{ICNm/WT} mouse and cross-correlation to purified alkaline phosphatase assay control, reveals the SeAP activity and serum phosphatase protein level to be non-linear; therefore, a calibration curve would be required to make definitive correlations between the SeAP activity and small, medium, or large cell masses secreting discrete levels of secreted alkaline phosphatase protein.

In order to evaluate the relative and absolute quantity of cells expressing the serological biomarker as a result of tamoxifen administration to Rosa26^{LUSAPm/WT} Pax7^{CreER/WT} Pax3^{P3Fm/WT} mice (wherein Pax3^{P3Fm/WT} represents an oncogenic mutation also called Pax3:Fkhr), alkaline phosphatase activity was measured from this animal and compared to a wild type mouse, a mouse with focal AAVcre activation in the right thigh, a MYF6cre mouse with Cre activation throughout the mature muscle and a HPRTcre mouse with ubiquitous Cre activation (FIG. 6C). The quantity of cells, as reflected by the serum alkaline phosphatase bioreporter, was intermediate between focal AAVcre activation and MYF6cre mature muscle cell levels (FIG. 6C). The absolute increases in SEAP activity are comparable to those reported in xenograft experiments of cells transfected with constitutively active SEAP expressing vectors (Bao et al., (2000), Gynecol Oncol, 78:373-379; Chaudhuri et al, (2003), Technol Cancer Res Treat, 2:171-180; Nilsson et al., (2002), Cancer Chemother Pharmacol, 49:93-100).

For comparison of signal to noise of the dual biomarker system to a red-shifted fluorescence reporter that is compatible with germline transmission, the fluorescence of the Z/RED red fluorescent protein reporter (RFP) mice with ubiquitous or no Cre activation was measured. Without shaving the fur of Z/RED-Tg^{GoZRED/W}^{_{T}} HPRT^{Cre/WT} ice, very little signal was seen except in the hairless areas of the paws, nose, and tail base. Over the abdomen where the animal was partially shaved, RFP signal could be observed with a dynamic range of 2.2×10¹ photons/cm²/sec/steradian signal over background.

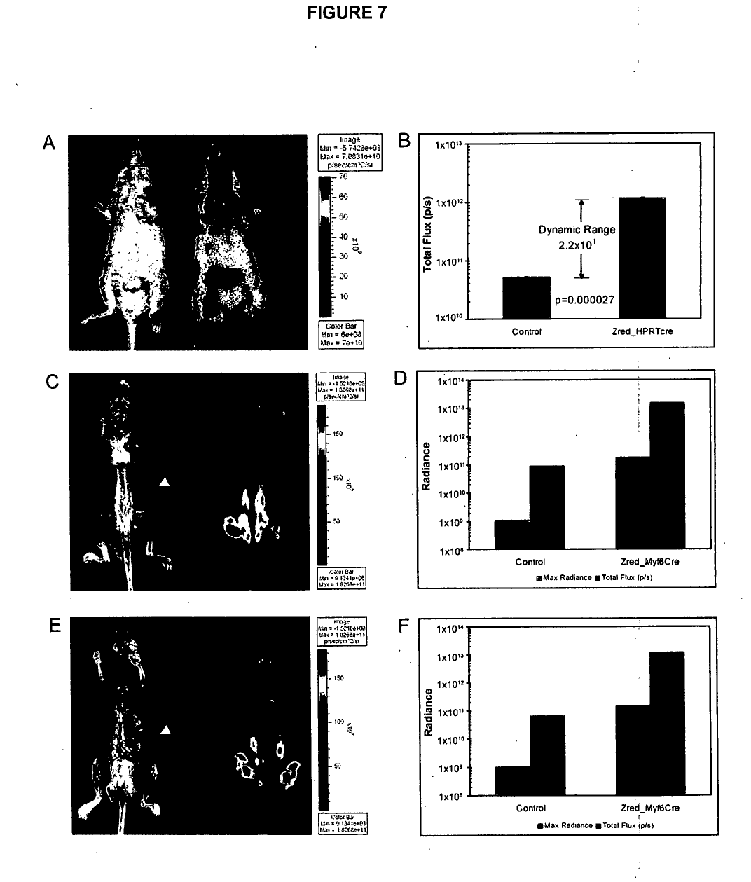

To investigate whether this signal might be from abdominal musculature or autofluorescent mouse chow, a sacrificed, denuded Z/RED-Tg^{GoZRED/WT} Myf6^{ICNm/WT} mouse with skeletal muscle expression of RFP was imaged (FIG. 7C-F), demonstrating fluorescent strong signal from rectus abdominis muscle. Very little autofluorescence was observed from mouse chow for control animals fed normal or purified mouse diets. Thus, the luciferase in the dual reporter system had more than a log better performance than RFP without the need for shaving, and the 2-log measurable range of serum SEAP was not affected by the fur or depth of signal.

### Example 6

### Generation of Tamoxifen-Inducible Satellite Cell Cre Driver Mouse Line

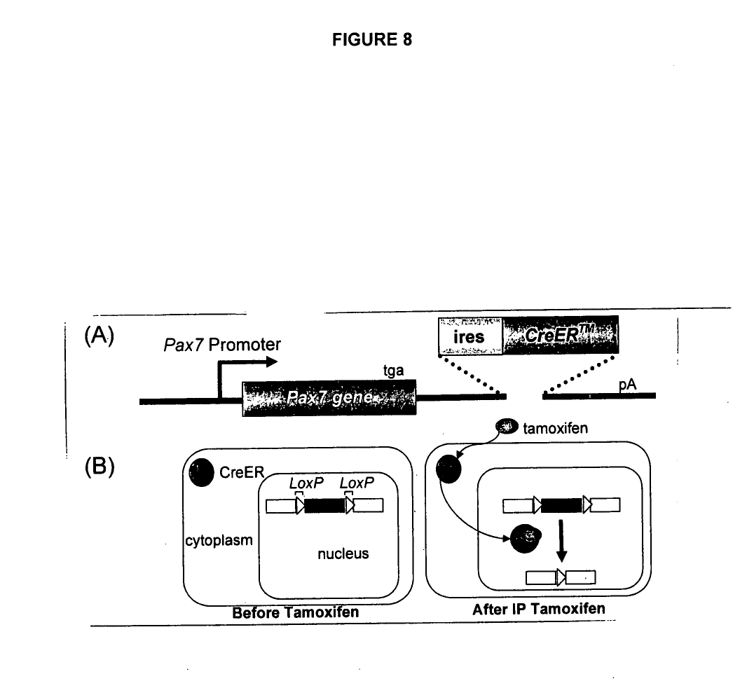

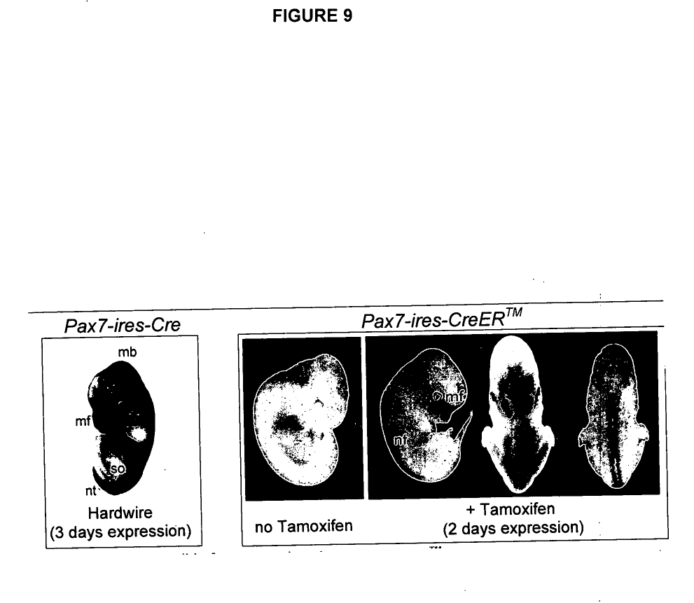

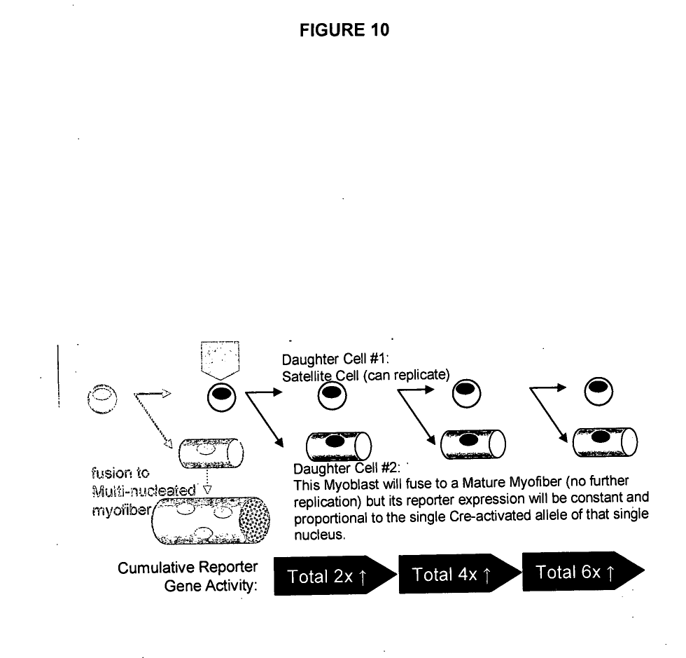

A mouse line expressing Cre in activated satellite cells expressing the Pax7 gene was generated using a Pax7-Cre driver with spatial and temporal selectivity. FIG. 8 illustrates the construct used in generating the transgenic mouse line. Cre is fused to the ligand binding domain of a tamoxifen-avid mutant estrogen receptor. Cre can be sequestered in the cytoplasm and kept inactive. When Cre was applied intraperitoneally to the mouse, the CreER fusion protein moved to the nucleus to find LoxP sites in the genomic DNA to rearrange. CreER is only active during the pulse of tamoxifen and does not remain active subsequent to the application of tamoxifen. Cre efficiency can vary with different mouse lines, but the DNA-rearranging actions of Cre are irreversible. The Pax7 CreER line used in the present experiments show virtually no background Cre activity (FIG. 9). In order to determine the detectable range of luminescent and serological biomarkers, the signal was compared between a wild type mouse, a mouse with Cre activation in the right thigh, a Myf6-Cre mouse with Cre activation throughout the mature muscle and a HPRT-Cre mouse with ubiquitous Cre activation. Quantitative luminescence (FIG. 9B) showed a dynamic range of nearly 2.5 logs of luminescence over background. Quantitative serum alkaline phosphatase activity (FIG. 9C) showed a dynamic range of more than 2 logs over background.

### Example 7

### Examination of Satellite Cell Dynamics in a Mouse Carrying an Oncogene

A Pax7-CreER/LUSAP dual reporter mouse line was used to examine the cell division kinetics of satellite cells expressing the oncogene Pax3:Fkhr. Pax3:Fkhr is a translocation-mediated chimeric fusion gene associated with the muscle cancer, alveolar rhabdomyosarcoma.

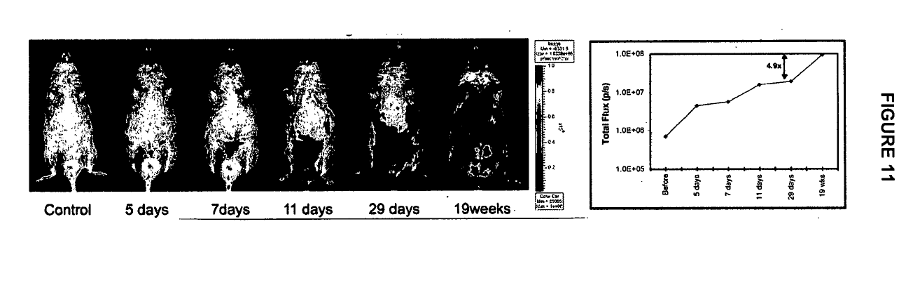

The mouse was given tamoxifen (7 mg/40 gm bodyweight) for five days. Induction of luciferase was seen as early as the fifth day after injection (FIG. 11). In the last three months of the experiment, the luciferase signal had increased 4.9 fold, consistent with 2.45 cell doublings, indicating satellite cell kinetics of approximately 1 cell division every 1.22 months. Quantitative measurement of absolute SEAP activity at the end of the fourth month was slightly higher than the activation SEAP level for focal AAVcre that is shown in FIG. 9.

### Example 8

### Luminescent Marking of Activated Satellite Cells and Serial Imaging

Serum alkaline phosphatase activity over time in aging mice is compared to activity of candidate biomarkers in a microarray gene expression analysis of serially sacrificed mice. Such a study identifies candidate biomarkers of age-related muscle wasting (sarcopenia) that correlate well with the decline in muscle stem cells that secrete the serum alkaline phosphatase BioReporter.

What is claimed:

1. A method of identifying a diagnostic biomarker in a subject expressing a BioReporter, said method comprising:
(a) comparing a first property of a candidate biomarker at a first time point with said first property of said candidate biomarker at a second time point, thereby determining a first comparison value for said first property of said candidate biomarker;
(b) comparing a first property of said BioReporter at said first time point with said first property of said BioReporter at said second time point, thereby determining a first comparison value for said first property of said BioReporter;
(c) comparing said first comparison value for said candidate biomarker with said first comparison value for said BioReporter to determine a first correlation for said first comparison value for said candidate biomarker and said first comparison value for said BioReporter; and
(d) comparing said first correlation with a reference first correlation, thereby identifying said candidate biomarker as a diagnostic biomarker.

2. A method according to claim 1 further comprising prior to step (a) determining said first property of said candidate biomarker at said first time point.

3. A method according to claim 1 further comprising prior to step (b) determining said first property of said BioReporter at said first time point.

4. A method according to claim 1, wherein said first property of said BioReporter comprises a detectable signal.

5. A method according to claim 4, wherein said detectable signal is an optical signal.

6. A method according to claim 5, wherein said optical signal is a fluorescent signal.

7. A method according to claim 4, wherein said detectable signal is a secreted molecule.

8. A method according to claim 7, wherein said secreted molecule is alkaline phosphatase.

9. A method according to claim 1, wherein said first property of said candidate biomarker comprises a level of expression.

10. A method according to claim 1, wherein said first property of said BioReporter comprises a level of expression.

11. A method according to claim 1, wherein said first property of said candidate biomarker comprises a pattern of tissue distribution.

12. A method according to claim 1, wherein said first property of said BioReporter comprises a pattern of tissue distribution.

13. A method according to claim 1, wherein said subject is a preclinical animal model.

14. A method according to claim 13, wherein said preclinical animal model is a member selected from: a LUSAP reporter mouse, a B2M mouse, and a UOX mouse.

15. A method according to claim 1, wherein said reference first correlation comprises a threshold value and wherein identifying said candidate biomarker as a diagnostic biomarker comprises determining whether said first correlation exceeds said threshold value.

16. A method according to claim 1, said method further comprising a validation step confirming said identifying said candidate biomarker as a diagnostic biomarker, said validation step comprising:
(i) comparing a second property of said candidate biomarker at a first time, point with said second property of said candidate biomarker at a second time point, thereby determining a second comparison value for said second property of said candidate biomarker;
(ii) comparing a second property of said BioReporter at said first time point with said second property of said BioReporter at said second time point, thereby determining a second comparison value for said second property of said BioReporter;
(iii) comparing said second comparison value for said candidate biomarker with said second comparison value for said BioReporter to determine a second correlation for said second comparison value for said candidate biomarker and said second comparison value for said BioReporter; and
(iv) comparing said second correlation with a reference second correlation, thereby confirming said identifying said candidate biomarker as a diagnostic biomarker.

17. A method according to claim 16, wherein said identifying said candidate biomarker as a diagnostic biomarker and said confirming said identifying said candidate biomarker as a diagnostic biomarker are accomplished essentially simultaneously.

18. A method according to claim 16, wherein said identifying said candidate biomarker as a diagnostic biomarker and said confirming said identifying said candidate biomarker as a diagnostic biomarker are accomplished sequentially.

19. A method according to claim 1, wherein said candidate biomarker is a member selected from a lipid, a phospholipid, a polypeptide, a glycoprotein, and a metabolite.

20. A method according to claim 19, wherein said polypeptide is a member selected from: a cell-surface bound polypeptide, a circulating polypeptide, and an intracellular polypeptide.

21. A method according to claim 1, said method further comprising determining a structure of said diagnostic biomarker.

22. A method of diagnosing a disease in a patient, said method comprising:
(a) determining a diagnostic biomarker property, wherein said diagnostic biomarker is identified by a method comprising:
i. comparing a first property of a candidate biomarker at a first time point with said first property of said candidate biomarker at a second time point, thereby determining a first comparison value for said first property of said candidate biomarker;
ii. comparing a first property of a BioReporter at said first time point with said first property of said BioReporter at said second time point, thereby determining a first comparison value for said first property of said BioReporter;
iii. comparing said first comparison value for said candidate biomarker with said first comparison value for said BioReporter to determine a first correlation for said first comparison value for said candidate biomarker and said first comparison value for said BioReporter; and
iv. comparing said first correlation with a reference first correlation, thereby identifying said candidate biomarker as a diagnostic biomarker;
(b) analyzing said diagnostic biomarker property, thereby determining a diagnostic biomarker property value;
(c) comparing said diagnostic biomarker property value to a reference diagnostic biomarker property value, thereby diagnosing a disease in said patient.

23. A method according to claim 22, wherein said disease is a member selected from: sarcopenia, cancer, neurodegenerative disease, and cardiovascular disease.

24. A method according to claim 22, wherein said analyzing said diagnostic biomarker property is a member selected from: measuring expression level of said diagnostic biomarker in said patient, and determining tissue localization of said diagnostic biomarker in said patient.

25. A method of determining effectiveness of a treatment for a disease in a patient, said method comprising:
(a) determining a diagnostic biomarker property, wherein said diagnostic biomarker is identified by a method comprising:
i. comparing a first property of a candidate biomarker at a first time point with said first property of said candidate biomarker at a second time point, thereby determining a first comparison value for said first property of said candidate biomarker;
ii. comparing a first property of a BioReporter at said first time point with said first property of said BioReporter at said second time point, thereby determining a first comparison value for said first property of said BioReporter;
iii. comparing said first comparison value for said candidate biomarker with said first comparison value for said BioReporter to determine a first correlation for said first comparison value for said candidate biomarker and said first comparison value for said BioReporter; and
iv. comparing said first correlation with a reference first correlation, thereby identifying said candidate biomarker as a diagnostic biomarker;
(b) analyzing said diagnostic biomarker property, thereby determining a diagnostic biomarker property value; and
(c) comparing said diagnostic biomarker property value to a reference diagnostic biomarker property value, thereby determining effectiveness of said treatment.

26. A method according to claim 25, wherein said disease is a member selected from: sarcopenia, cancer, neurodegenerative disease, and cardiovascular disease.

27. A method according to claim 25, wherein said analyzing said diagnostic biomarker property is a member selected from: measuring expression level of said diagnostic biomarker in said patient, and determining tissue localization of said diagnostic biomarker in said patient.

28. A method of identifying a diagnostic biomarker in a subject, said method comprising:
(a) inducing expression of a BioReporter in said subject;
(b) comparing a first property of a candidate biomarker at a first time point with said first property of said candidate biomarker at a second time point, thereby determining a first comparison value for said first property of said candidate biomarker;
(c) comparing a first property of said BioReporter at said first time point with said first property of said BioReporter at a second time point, thereby determining a first comparison value for said first property of said BioReporter;
(d) comparing said first comparison value for said candidate biomarker with said first comparison value for said BioReporter to determine a first correlation for said first comparison value for said candidate biomarker and said first comparison value for said BioReporter; and
(e) comparing said first correlation with a reference first correlation, thereby identifying said candidate biomarker as a diagnostic biomarker.

29. A method according to claim 28, wherein said inducing expression of a BioReporter comprises:
(a) modifying a cell to express said BioReporter, and
(b) incorporating said cell into said subject, thereby inducing expression of said BioReporter.

30. A kit which comprises:
(a) an isolated diagnostic biomarker, wherein said isolated diagnostic biomarker is prepared by a method comprising isolating said diagnostic biomarker, wherein prior to said isolating, said diagnostic biomarker is determined to be a diagnostic biomarker by a method comprising
i. comparing a first property of a candidate biomarker at a first time point with said first property of said candidate biomarker at a second time point, thereby determining a first comparison value for said first property of said candidate biomarker;
ii. comparing a first property of said BioReporter at said first time point with said first property of said BioReporter at said second time point, thereby determining a first comparison value for said first property of said BioReporter;
iii. comparing said first comparison value for said candidate biomarker with said first comparison value for said BioReporter to determine a first correlation for said first comparison value for said candidate biomarker and said first comparison value for said BioReporter; and
iv. comparing said first correlation with a reference first correlation, thereby identifying said candidate biomarker as a diagnostic biomarker;
(b) a container, wherein said container comprises said isolated diagnostic biomarker; and
(c) instructions for using said diagnostic biomarker in a method which is a member selected from: diagnosing a disease, determining effectiveness of a treatment, and identifying causative factors of a disease.
